(19) United States
(12) Patent Application Publication
(10) Pub. No.: US 2008/0069796 A1
(43) Pub. Date: Mar. 20, 2008

(54) Low Dose Treatment with an Interleukin-11 Analog

(76) Inventors: Jong-Mook Kim, Seoul (KR); Jae-Gyun Jeong, Seoul (KR); Song Shan Xu, Beijing (CN)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/830,993

(22) Filed: Jul. 31, 2007

Related U.S. Application Data
(60) Provisional application No. 60/834,167, filed on Jul. 31, 2006.

(51) Int. Cl. A61K 38/20; A61P 37/00

(52) U.S. Cl. 424/85.2

(57) ABSTRACT

The invention relates to an analog of human interleukin-11 (IL-11) polypeptide which has increased potency and stability relative to wild-type IL-11 and which is therapeutically effective at low doses.

This application claims the benefit of priority of U.S. Provisional Application No. 60/834,167, filed Jul. 31, 2006, hereby incorporated by reference.

### BACKGROUND OF THE INVENTION

1. Field of the Invention

This invention is in the field of biological therapeutics. In particular, the invention relates to an analog of human interleukin-11 (IL-11) which has increased potency and stability relative to wild-type IL-11 and which is therapeutically effective at low doses.

2. Related Art

IL-11 is a stromal cell-derived cytokine which interacts with a variety of hematopoietic and non-hematopoietic cell types. IL-11 plays a major role in the differentiation of stem cells into megakaryocytes, the proliferation and maturation of megakaryocytes, and the generation of platelets.

Recombinant human IL-11, marketed by Wyeth-Ayerst as NEUMEGA® (generic name oprelvekin), is approved for the prevention of severe thrombocytopenia and the reduction of the need for platelet transfusions following myelosuppressive chemotherapy in adult patients with nonmyeloid malignancies who are at high risk of severe thrombocytopenia. NEUMEGA® is supplied in a single use vial containing 5 mg IL-11 as a lyophilized powder. The powder is reconstituted with 1 mL Sterile Water for Injection, USP to produce a solution comprising 5 mg/mL IL-11 and is administered at a dose of 50 μg/kg/day. The most frequent adverse events associated with NEUMEGA® include atrial arrhythmias, syncope, dyspnea, congestive heart failure, and pulmonary edema.

Chinese Patent No. 11677 discloses an analog of human IL-11 (SEQ ID NO:1) in which the first nine amino acids of the mature polypeptide have been deleted, Val¹⁰ has been changed to Ala¹⁰, and Asp¹³⁴ has been changed to Asn¹³⁴. This analog displays enhanced resistance to acidolysis and increased stability compared to wild-type IL-11.

### SUMMARY OF THE INVENTION

The present invention relates to an analog of human IL-11 polypeptide comprising the amino acid sequence of SEQ ID NO:1 or a fragment or derivative thereof which may be administered to animals in need of IL-11 at lower doses than that used for wild-type IL-11, thereby avoiding or minimizing side effects due to IL-11 treatment. Alternatively, the IL-11 analog may be administered at doses typically used for wild-type IL-11 but with an enhanced effect relative to wild-type IL-11.

Thus, one embodiment of the present invention relates to methods for treating, ameliorating, or preventing a disease or disorder responsive to IL-11 in an animal, comprising administering to said animal an IL-11 analog comprising the amino acid sequence of SEQ ID NO:1 or a fragment or derivative thereof at a low dose, e.g., about 3 to about 20 μg/kg/day.

Another embodiment of the present invention relates to methods for stimulating thrombopoiesis in an animal in need thereof, comprising administering to said animal an IL-11 analog comprising the amino acid sequence of SEQ ID NO:1 or a fragment or derivative thereof at a low dose, e.g., about 3 to about 20 μg/kg/day.

A further embodiment of the present invention relates to methods for increasing the platelet count in an animal in need thereof, comprising administering to said animal an IL-11 analog comprising the amino acid sequence of SEQ ID NO:1 or a fragment or derivative thereof at a low dose, e.g., about 3 to about 20 μg/kg/day.

The present invention further relates to pharmaceutical compositions comprising an IL-11 analog comprising the amino acid sequence of SEQ ID NO:1 or a fragment or derivative thereof and a pharmaceutically acceptable carrier. In certain embodiments, the pharmaceutical compositions are in unit dosage form and comprise a low dose of IL-11 analog or a fragment or derivative thereof, e.g., less than about 4.5 mg.

The present invention further relates to kits comprising an IL-11 analog comprising the amino acid sequence of SEQ ID NO:1 or a fragment or derivative thereof. In one embodiment the IL-11 analog is in the form of a pharmaceutical composition comprising the IL-11 analog and a pharmaceutically acceptable carrier. In certain embodiments, the pharmaceutical compositions are in unit dosage form and comprise a low dose of IL-11 analog or a fragment or derivative thereof, e.g., less than about 4.5 mg.

Another embodiment of the present invention relates to methods for preparing a pharmaceutical composition comprising an IL-11 analog comprising the amino acid sequence of SEQ ID NO:1 or a fragment or derivative thereof, the method comprising admixing said IL-11 analog or a fragment or derivative thereof and a pharmaceutically acceptable carrier.

### BRIEF DESCRIPTION OF THE DRAWINGS/FIGURES

FIG. 1 shows the increase in blood platelet counts in response to administration of IL-11 analog and NEUMEGA® in cynomolgus monkeys.

### DETAILED DESCRIPTION OF THE INVENTION

The present invention relates to the use of an analog of human IL-11 comprising the amino acid sequence of SEQ ID NO:1 or a fragment or derivative thereof for the treatment, amelioration, or prevention of diseases or disorders responsive to IL-11. The amino acid sequence of the IL-11 analog is listed below.

(SEQ ID NO:1)

Ala-Ser-Pro-Asp-Pro-Arg-Ala-Glu-Leu-Asp-Ser-Thr-
Val-Leu-Leu-Thr-Arg-Ser-Leu-Leu-Ala-Asp-Thr-Arg-
Gln-Leu-Ala-Ala-Gln-Leu-Arg-Asp-Lys-Phe-Pro-Ala-
Asp-Gly-Asp-His-Asn-Leu-Asp-Ser-Leu-Pro-Thr-Leu-
Ala-Met-Ser-Ala-Gly-Ala-Leu-Gly-Ala-Leu-Gln-Leu-
Pro-Gly-Val-Leu-Thr-Arg-Leu-Arg-Ala-Asp-Leu-Leu-
Ser-Tyr-Leu-Arg-His-Val-Gln-Trp-Leu-Arg-Arg-Ala-
Gly-Gly-Ser-Ser-Leu-Lys-Thr-Leu-Glu-Pro-Glu-Leu-
Gly-Thr-Leu-Gln-Ala-Arg-Leu-Asp-Arg-Leu-Leu-Arg-
Arg-Leu-Gln-Leu-Leu-Met-Ser-Arg-Leu-Ala-Leu-Pro-
Gln-Pro-Pro-Pro-Asn-Pro-Pro-Ala-Pro-Pro-Leu-Ala-
Pro-Pro-Ser-Ser-Ala-Try-Gly-Gly-Ile-Arg-Ala-Ala-
His-Ala-Ile-Leu-Gly-Gly-Leu-His-Leu-Thr-Leu-Asp-
Trp-Ala-Val-Arg-Gly-Leu-Leu-Leu-Leu-Lys-Thr-Arg-
Leu

The term “IL-11 analog,” as used herein, refers to a polypeptide comprising the amino acid sequence of SEQ ID NO:1.

The term “fragment thereof,” as applied to the IL-11 analog, refers to a polypeptide comprising any portion of the amino acid sequence of the IL-11 analog, wherein the fragment retains substantially all of the biological activity of the full-length IL-11 analog. The term fragment includes internal deletions of the IL-11 analog as well as truncations of the N and/or C terminus of the polypeptide. In one embodiment, the fragment is a truncation from the N-terminal end of the polypeptide. In another embodiment, the fragment retains one or both of Ala¹⁰ and Asn¹³⁴ of the IL-11 analog (corresponding to positions 1 and 125 of SEQ ID NO:1). In one embodiment, the fragment retains Ala¹⁰. In another embodiment, the fragment retains Asn¹³⁴.

In other embodiments, the IL-11 analog comprises a polypeptide comprising amino acids x to 169 of SEQ ID NO:1, wherein x is any integer from 1 to 40, and wherein said analog retains substantially all of the biological activity of the full-length IL-11 analog. In other embodiments, x is an integer from 1 to 25. In other embodiments, x is an integer from 1 to 10. In another embodiment, x is an integer from 1 to 5.

In other embodiments, the IL-11 analog comprises a polypeptide comprising amino acids 1 to y of SEQ ID NO:1, wherein in y is any integer from 125 to 169, and wherein said analog retains substantially all of the biological activity of the full-length IL-11 analog. In certain embodiments, y is an integer from 140 to 169. In other embodiments, y is an integer from 160 to 169. In another embodiment, y is an integer from 165 to 169. In another embodiment, y is 125.

In other embodiments, the IL-11 analog comprises a polypeptide comprising amino acids x to y of SEQ ID NO:1, wherein x is any integer from 1 to 40 and y is any integer from 125 to 169, and wherein said analog retains substantially all of the biological activity of the full-length IL-11 analog. In further embodiments, x is any integer from 1 to 25. In other embodiments, x is an integer from 1 to 10. In another embodiment, x is an integer from 1 to 5. In other embodiments, y is an integer from 140 to 169. In other embodiments, y is an integer from 160 to 169. In another embodiment, y is an integer from 165 to 169. In another embodiment, y is 125.

The term “derivative thereof,” as applied to the IL-11 analog, refers to a polypeptide consisting of an amino acid sequence that is at least 70% identical to the amino acid sequence of SEQ ID NO:1, wherein the polypeptide retains substantially all of the biological activity of the IL-11 analog. In some embodiments, the derivative comprises an amino acid sequence that is at least 75%, 80%, 85%, 90%, 95%, 96%, 97%, 98%, or 99% identical to the amino acid sequence of SEQ ID NO:1. In another embodiment, the derivative retains one or both of Ala¹⁰ and Asn¹³⁴ of the IL-11 analog (corresponding to positions 1 and 125 of SEQ ID NO:1). In one embodiment, the derivative retains Ala¹⁰. In another embodiment, the derivative retains Asn¹³⁴. The derivative may comprise additions, deletions, substitutions, or a combination thereof to the amino acid sequence of SEQ ID NO:1. Additions or substitutions also include the use of non-naturally occurring amino acids and may occur in any number internally, at the N-terminus and/or the C-terminus, so long as the polypeptide retains substantially all of the biological activity of the IL-11 analog.

Preferably, any substitutions are conservative amino acid substitutions. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Sequence identity is calculated by comparing two optimally aligned sequences over that region of comparison, determining the number of positions at which the identical amino acid residue occurs in both sequences to yield the number of matched positions, dividing the number of matched positions by the total number of positions in the region of comparison (i.e., the window size), and multiplying the result by 100 to yield the percentage of sequence identity. In one aspect, percent identity is calculated as the percentage of amino acid residues in the smaller of two sequences which align with an identical amino acid residue in the sequence being compared, when four gaps in a length of 100 amino acids may be introduced to maximize aligmnent (Dayhoff, in Atlas of Protein Sequence and Structure, Vol. 5, p. 124, National Biochemical Research Foundation, Washington, D.C. (1972), incorporated herein by reference). A determination of identity is typically made by a computer homology program known in the art. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

The structure and function of human IL-11 has been extensively studied and one of skill in the art is aware of the amino acids in the IL-11 sequence that are important for retaining substantially all of the biological activity of the protein and that are preferably not changed or only conservatively changed in any derivative of the IL-11 analog. Other amino acids that are not critical to biological activity may be deleted and/or substituted more freely. Examples of amino acids known to be important for biological activity include, but are not limited to, Met⁵⁹, Lys⁴¹, Lys⁹⁸, Lys¹⁷⁵, Thr¹⁷⁶, Arg¹⁷⁷, and Lys¹⁷⁸ (Czupryn et al., J. Biol. Chem. 270:978 (1995)); Pro¹⁴, Asp¹⁷, Leu¹⁸, Leu²³, Arg²⁶, Leu²⁹, Thr³², Arg³³, Leu³⁵, Arg⁴⁰, Ala¹⁵³, Ile¹⁵⁶, Gy¹⁵⁹, Leu¹⁶⁰, Thr¹⁶³, Leu¹⁷¹, Arg¹⁵¹, Met¹⁵⁴, Glu¹⁶⁵, Trp¹⁶⁶ and Arg¹⁶⁹ (Czupryn et al., Ann. NY Acad. Sci. 762:152 (1995)); Leu⁶⁴, Ile¹⁵⁰, Arg¹⁶⁹, and Leu¹⁷³ (Tacken et al., Eur. J. Biochem. 265:645 (1999)). One of skill in the art can prepare derivatives of the IL-11 analog using routine mutagenesis techniques, such as those described in the references cited above, and identify derivatives retaining substantially all of the biological activity of the IL-11 analog.

The term “substantially all of the biological activity of the IL-11 analog,” as used herein, refers to a fragment or derivative of the IL-11 analog which retains at least 70% of any one or more of the biological activities of the IL-11 analog (e.g., the ability to stimulate thrombopoiesis or other recognized biological activities of IL-11). In some embodiments, at least 75, 80, 85, 90, or 95% of one or more of the biological activities of the IL-11 analog is retained. IL-11 activity may be determined by routine in vitro and in vivo assays well known in the art (e.g., megakaryocyte proliferation assay, spleen cell plaque formation assay, stimulation of platelet blood levels).

In some embodiments of the present invention, an isolated or purified IL-11 analog or a fragment or derivative thereof is used in the methods of the invention. An “isolated” or “purified” protein thereof is substantially free of cellular material or other contaminating proteins from the cell or tissue source from which the IL-11 analog protein is derived, or substantially free from chemical precursors or other chemicals when chemically synthesized. The language “substantially free of cellular material” includes preparations of the IL-11 analog protein in which the protein is separated from cellular components of the cells from which it is recombinantly produced. In one embodiment, the language “substantially free of cellular material” includes preparations of the IL-11 analog protein having less than about 30% (by dry weight) of non-IL-11 analog protein (also referred to herein as a “contaminating protein”), e.g., less than about 20%, less than about 10%, or less than about 5% of non-IL-11 analog protein. When the IL-11 analog protein or a fragment or derivative thereof is recombinantly produced, it is also preferably substantially free of culture medium, i.e., culture medium represents less than about 20%, e.g., less than about 10% or less than about 5% of the volume of the protein preparation.

The language “substantially free of chemical precursors or other chemicals” includes preparations of the IL-11 analog protein or a fragment or derivative thereof in which the protein is separated from chemical precursors or other chemicals that are involved in the synthesis of the protein. In one embodiment, the language “substantially free of chemical precursors or other chemicals” includes preparations of the IL-11 analog protein having less than about 30% (by dry weight) of chemical precursors or non-IL-11 analog chemicals, e.g., less than about 20%, less than about 10%, or less than about 5% chemical precursors or non-IL-11 analog chemicals.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of thrombocytopenia, a therapeutically effective amount preferably refers to the amount of a therapeutic agent that increases the blood level of platelets by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “prevent,” “preventing,” and “prevention,” as used herein, refer to a decrease in the occurrence of pathological cells or a lack of decrease in desirable cells (e.g., platelets) in an animal. The prevention may be complete, e.g., the total absence of a decrease in desirable cells in a subject. The prevention may also be partial, such that the decrease in desirable cells in a subject is less than that which would have occurred without the present invention.

The IL-11 analog or a fragment or derivative thereof may be produced by any method known in the art, e.g., recombinant expression or chemical synthesis. Preferably, the IL-11 is recombinantly expressed, e.g., in bacterial, yeast, or mammalian cell cultures. Recombinant expression involves preparing a vector comprising a polynucleotide encoding the IL-11 analog, delivering the vector into a host cell, culturing the host cell under conditions in which the IL-11 analog is expressed, and separating the IL-11 analog. Methods and materials for preparing recombinant vectors and transforming host cells using the same, replicating the vectors in host cells and expressing biologically active foreign polypeptides and proteins are described in Sambrook et al., Molecular Cloning, 3rd edition, Cold Spring Harbor Laboratory, 2001 and Ausubel et al., Current Protocols in Molecular Biology, John Wiley & Sons, New York 3rd edition, (2000), each incorporated herein by reference. Derivatives of the IL-11 analog may be produced using routine mutagenesis techniques well known in the art (see, e.g., U.S. Pat. No. 4,518,584).

The IL-11 analog amino acid sequence information may be used to create a polynucleotide sequence encoding the IL-11 analog or a fragment or derivative thereof. The polynucleotide sequence may be chemically synthesized or derived from a gene or cDNA encoding wild-type IL-11. The availability of the polynucleotide sequence encoding the IL-11 analog makes possible large-scale expression of the encoded polypeptide by techniques well known and routinely practiced in the art.

Vectors are used herein either to amplify DNA or RNA encoding the IL-11 analog and/or to express DNA which encodes the IL-11 analog. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), that serve equivalent functions.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith et al., Gene 67:31-40 (1988)), pMAL (New England Biolabs, Beverly, Mass.) and pRIT5 (Pharmacia, Piscataway, N.J.) that fuse glutathione-S-transferase (GST), maltose E binding protein, or protein A, respectively, to the target recombinant protein.

Examples of suitable inducible non-fusion E. coli expression vectors include pTrc (Amrann et al., Gene 69:301-315 (1988)) and pET 1 Id (Studier et al., GENE EXPRESSION TECHNOLOGY: METHODS IN ENZYMOLOGY 185, Academic Press, San Diego, Calif. (1990) 60-89). One strategy to maximize recombinant protein expression in E. coli is to express the protein in host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., Nuc. Acids Res. 20:2111-2118 (1992)). Such alteration of nucleic acid sequences of the invention can be carried out by standard DNA synthesis techniques.

In another embodiment, the IL-11 analog expression vector is a yeast expression vector. Examples of vectors for expression in yeast S. cerivisae include pYepSecl (Baldari et al., EMBO J. 6:229-234 (1987)), pMFa (Kurjan et al., Cell 30:933-943 (1982)), pJRY88 (Schultz et al., Gene 54:113-123 (1987)), pYES2 (Invitrogen Corporation, San Diego, Calif.), and picZ (InVitrogen Corp, San Diego, Calif.).

Alternatively, the IL-11 analog can be expressed in insect cells using baculovirus expression vectors. Baculovirus vectors available for expression of proteins in cultured insect cells (e.g., SF9 cells) include the pAc series (Smith et al., Mol. Cell. Biol. 3:2156-2165 (1983)) and the pVL series (Lucklow et al., Virology 170:31-39 (1989)).

In yet another embodiment, a nucleic acid of the invention is expressed in mammalian cells using a mammalian expression vector. Examples of mammalian expression vectors include pCDM8 (Seed, Nature 329:840 (1987)) and pMT2PC (Kaufman et al., EMBO J. 6: 187-195 (1987)). When used in mammalian cells, the expression vector's control functions are often provided by viral regulatory elements. For example, commonly used promoters are derived from polyoma, adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. Genes Dev. 1:268-277 (1987)), lymphoid-specific promoters (Calame et al., Adv. Immunol. 43:235-275 (1988)), in particular promoters of T cell receptors (Winoto et al., EMBO J. 8:729-733 (1989)) and immunoglobulins (Banerji et al., Cell 33:729-740 (1983); Queen et al., Cell 33:741-748 (1983)), neuron-specific promoters (e.g., the neurofilament promoter; Byrne et al., Proc. Natl. Acad. Sci. USA 86:5473-5477 (1989)), pancreas-specific promoters (Edlund et al., Science 230:912-916 (1985)), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel et al., Science 249:374-379 (1990)) and the α-fetoprotein promoter (Campes et al., Genes Dev. 3:537-546 (1989)).

Preferred vectors include, but are not limited to, plasmids, phages, cosmids, episomes, viral particles or viruses, and integratable DNA fragments (i.e., fragments integratable into the host genome by homologous recombination). Preferred viral particles include, but are not limited to, adenoviruses, baculoviruses, parvoviruses, herpesviruses, poxviruses, adenoassociated viruses, Semliki Forest viruses, vaccinia viruses, and retroviruses. Preferred expression vectors include, but are not limited to, pcDNA3 (Invitrogen) and pSVL (Pharmacia Biotech). Other expression vectors include, but are not limited to, pSPORT™ vectors, pGEM™ vectors (Promega), pPROEXvectors™ (LTI, Bethesda, Md.), Bluescript™ vectors (Stratagene), pQE™ vectors (Qiagen), pSE420™ (Invitrogen), and pYES2™ (Invitrogen).

Preferred expression vectors are replicable DNA constructs in which a DNA sequence encoding the IL-11 analog is operably linked or connected to suitable control sequences capable of effecting the expression of the IL-11 analog in a suitable host. DNA regions are operably linked or connected when they are functionally related to each other. For example, a promoter is operably linked or connected to a coding sequence if it controls the transcription of the sequence. Amplification vectors do not require expression control domains, but rather need only the ability to replicate in a host, usually conferred by an origin of replication, and a selection gene to facilitate recognition of transformants. The need for control sequences in the expression vector will vary depending upon the host selected and the transformation method chosen. Generally, control sequences include, but are not limited to a transcriptional promoter, enhancers, an optional operator sequence to control transcription, polyadenylation signals, a sequence encoding suitable mRNA ribosomal binding and sequences which control the termination of transcription and translation. Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein. Preferred vectors preferably contain a promoter that is recognized by the host organism. The promoter sequences of the present invention may be prokaryotic, eukaryotic or viral. Examples of suitable prokaryotic sequences include the PR and PL promoters of bacteriophage lambda (The bacteriophage Lambda, Hershey, A. D., Ed., Cold Spring Harbor Press, Cold Spring Harbor, N.Y. (1973), which is incorporated herein by reference in its entirety; Lambda II, Hendrix, R. W., Ed., Cold Spring Harbor Press, Cold Spring Harbor, N.Y. (1980), which is incorporated herein by reference in its entirety); the trp, recA, heat shock, and lacZ promoters of E. coli and the SV40 early promoter (Benoist et al., Nature, 290:304-310 (1981)), which is incorporated herein by reference in its entirety). Additional promoters include, but are not limited to, mouse mammary tumor virus, long terminal repeat of human immunodeficiency virus, maloney virus, cytomegalovirus immediate early promoter, Epstein Barr virus, Rous sarcoma virus, human actin, human myosin, human hemoglobin, human muscle creatine, and human metalothionein.

Additional regulatory sequences can also be included in preferred vectors. Preferred examples of suitable regulatory sequences are represented by the Shine-Dalgarno sequence of the replicase gene of the phage MS-2 and of the gene cII of bacteriophage lambda. The Shine-Dalgarno sequence may be directly followed by DNA encoding the IL-11 analog and result in the expression of the IL-11 analog protein.

Moreover, suitable expression vectors can include an appropriate marker that allows the screening of the transformed host cells. The transformation of the selected host is carried out using any one of the various techniques well known to the expert in the art and described in Sambrook et al., supra.

An origin of replication can also be provided either by construction of the vector to include an exogenous origin or may be provided by the host cell chromosomal replication mechanism. If the vector is integrated into the host cell chromosome, the latter may be sufficient. Alternatively, rather than using vectors which contain viral origins of replication, one skilled in the art can transform mammalian cells by the method of co-transformation with a selectable marker and IL-11 analog DNA. An example of a suitable marker is dihydrofolate reductase (DHFR) or thymidine kinase (see, U.S. Pat. No. 4,399,216).

Nucleotide sequences encoding the IL-11 analog may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesiderable joining, and ligation with appropriate ligases. Techniques for such manipulation are disclosed by Sambrook et al., supra and are well known in the art. Methods for construction of mammalian expression vectors are disclosed in, for example, Okayama et al., Mol. Cell. Biol. 3:280 (1983), Cosman et al., Mol. Immunol. 23:935 (1986), Cosman et al., Nature 312:768 (1984), EP-A-0367566, and WO 91/18982, each of which is incorporated herein by reference in its entirety.

According to another aspect of the invention, host cells are provided, including prokaryotic and eukaryotic cells, comprising a polynucleotide encoding the IL-11 analog in a manner that permits expression of the encoded IL-11 analog polypeptide. Polynucleotides of the invention may be introduced into the host cell as part of a circular plasmid, or as linear DNA comprising an isolated protein coding region or a viral vector. Methods for introducing DNA into the host cell that are well known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, vertebrate, and mammalian cells systems.

Host cells of the invention are useful in methods for the large-scale production of the IL-11 analog polypeptides wherein the cells are grown in a suitable culture medium and the desired polypeptide products are isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

Suitable host cells for expression of the polypeptides of the invention include, but are not limited to, prokaryotes, yeast, and eukaryotes. If a prokaryotic expression vector is employed, then the appropriate host cell would be any prokaryotic cell capable of expressing the cloned sequences. Suitable prokaryotic cells include, but are not limited to, bacteria of the genera Escherichia, Bacillus, Salmonella, Pseudomonas, Streptomyces, and Staphylococcus.

If a eukaryotic expression vector is employed, then the appropriate host cell would be any eukaryotic cell capable of expressing the cloned sequence. Preferably, eukaryotic cells are cells of higher eukaryotes. Suitable eukaryotic cells include, but are not limited to, non-human mammalian tissue culture cells and human tissue culture cells. Preferred host cells include, but are not limited to, insect cells, HeLa cells, Chinese hamster ovary cells (CHO cells), African green monkey kidney cells (COS cells), human 293 cells, and murine 3T3 fibroblasts. Propagation of such cells in cell culture has become a routine procedure (see, Tissue Culture, Academic Press, Kruse and Patterson, Eds. (1973), which is incorporated herein by reference in its entirety).

In addition, a yeast host may be employed as a host cell. Preferred yeast cells include, but are not limited to, the genera Saccharomyces, Pichia, and Kluveromyces. Preferred yeast hosts are S. cerevisiae and P. pastoris. Preferred yeast vectors can contain an origin of replication sequence from a 2T yeast plasmid, an autonomously replication sequence (ARS), a promoter region, sequences for polyadenylation, sequences for transcription termination, and a selectable marker gene. Shuttle vectors for replication in both yeast and E. coli are also included herein.

Alternatively, insect cells may be used as host cells. In a preferred embodiment, the polypeptides of the invention are expressed using a baculovirus expression system (see, Luckow et al., Bio/Technology, 6:47 (1988), BACULOVIRUS EXPRESSION VECTORS: A LABORATORY MANUAL, O'Rielly et al. (Eds.), W. H. Freeman and Company, New York, 1992, and U.S. Pat. No. 4,879,236, each of which is incorporated herein by reference in its entirety). In addition, the MAXBAC™ complete baculovirus expression system (Invitrogen) can, for example, be used for production in insect cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, dihydrofolate reductase (DHFR) and methotrexate. Nucleic acid encoding a selectable marker can be introduced into a host cell on the same vector as that encoding the IL-11 analog or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the IL-11 analog protein. In one embodiment, the method comprises culturing the host cell of invention (into which a recombinant expression vector encoding the IL-11 analog has been introduced) in a suitable medium such that the IL-11 analog protein is produced. In another embodiment, the method further comprises isolating the IL-11 analog from the medium or the host cell.

In situations where the IL-11 analog polypeptide will be found primarily intracellularly, intracellular material (including inclusion bodies for Gram-negative bacteria) can be extracted from the host cell using any standard technique known to one of ordinary skill in the art. Such methods would encompass, by way of example and not by way of limitation, lysing the host cells to release the contents of the periplasm/cytoplasm by French press, homogenization, and/or sonication followed by centrifugation.

If the IL-11 analog polypeptide has formed inclusion bodies in the cytosol, such inclusion bodies may frequently bind to the inner and/or outer cellular membranes. Upon centrifugation, the inclusion bodies will be found primarily in the pellet material. The pellet material can then be treated at pH extremes or with one or more chaotropic agents such as a detergent, guanidine, guanidine derivatives, urea, or urea derivatives in the presence of a reducing agent such as dithiothreitol at alkaline pH or tris-carboxyethyl phosphine at acid pH to release, break apart, and solubilize the inclusion bodies. Once solubilized, the IL-11 analog polypeptide can be analyzed using gel electrophoresis, immunoprecipitation or the like. Various methods of isolating the IL-11 analog polypeptide would be apparent to one of ordinary skill in the art, for example, isolation may be accomplished using standard methods such as those set forth below and in Marston et al., Meth. Enzymol. 182:264-275 (1990) (incorporated by reference herein in its entirety).

If isolated IL-11 analog polypeptide is not biologically active following the isolation procedure employed, various methods for “refolding” or converting the polypeptide to its tertiary structure and generating disulfide linkages, can be used to restore biological activity. Methods known to one of ordinary skill in the art include adjusting the pH of the solubilized polypeptide to a pH usually above 7 and in the presence of a particular concentration of a chaotrope. The selection of chaotrope is very similar to the choices used for inclusion body solubilization but usually at a lower concentration and is not necessarily the same chaotrope as used for the solubilization. It may be required to employ a reducing agent or the reducing agent plus its oxidized form in a specific ratio, to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol (DTT)/dithiane DTT, 2-mercaptoethanol (bME)/dithio-b(ME). To increase the efficiency of the refolding, it may be necessary to employ a cosolvent, such as glycerol, polyethylene glycol of various molecular weights, and arginine.

The present invention encompasses methods of treating, ameliorating, or preventing diseases or disorders that are responsive to IL-11. Examples of diseases or disorders that may be responsive to IL-11 administration include, but are not limited to, thrombocytopenia (e.g., induced by myelosuppressive chemotherapy), immune-mediated disorders (e.g., cytotoxic T cell- and complement-mediated cytotoxicity, graft-versus-host disease), mucositis (e.g., oral mucositis, gastrointestinal mucositis, nasal mucositis, proctitis), inflammatory bowel diseases (e.g., Crohn's disease, ulcerative colitis, indeterminate colitis, infectious colitis), inflammatory skin disorders (e.g., psoriasis, atopic dermatitis, contact hypersensitivity), gingivitis, periodontitis, ocular inflammatory diseases (e.g., conjunctivitis, retinitis, uveitis), gastrointestinal motility disorders (e.g., gastroesophageal reflux disease, feeding intolerance, post-operative adynamic ileus), pancreatitis, necrotizing enterocolitis, aphthous ulcers, pharyngitis, esophagitis, peptic ulcers, AIDS, rheumatoid arthritis, osteoarthritis, spondyloarthropathies, antibiotic-induced diarrheal diseases, multiple sclerosis, diabetes, osteoporosis, reperfusion injuries, asthma, rhinitis, preeclampsia, Von Willebrand disease, Non-Hodgkins lymphoma, and hematopoietic progenitor or stem cell deficiencies.

The IL-11 analog or a fragment or derivative thereof may be administered after the onset of symptoms of a disease or disorder. In other embodiments, the IL-11 analog or a fragment or derivative thereof may be administered prior to the onset of a disease or disorder in situations in which the disease or disorder is likely to occur in order to prevent or reduce the severity of the disease or disorder. For example, the IL-11 analog or a fragment or derivative thereof may be administered to a patient undergoing a chemotherapy treatment that is known to cause thrombocytopenia.

The IL-11 analog or a fragment or derivative thereof may be administered in combination with one or more other therapeutic agents or treatments known to be effective for the treatment, amelioration, or prevention of a disease or disorder. Examples of other therapeutic agents or treatments include, without limitation, other growth factors (e.g., interleukins, interferons, colony stimulating factors, tumor necrosis factors, erythropoietin), immunosuppressive agents, anti-inflammatory agents, anti-cancer agents, antibodies, and radiation. The IL-11 analog or a fragment or derivative thereof and one or more therapeutic agents may be administered as a single composition or as separate compositions. In some embodiments, the IL-11 analog or a fragment or derivative thereof and one or more therapeutic agents are administered to an animal under one or more of the following conditions: at different periodicities, at different durations, at different concentrations, by different administration routes, etc. In some embodiments, the IL-11 analog or a fragment or derivative thereof is administered prior to the therapeutic agent, e.g., 0.5, 1,2, 3,4, 5, 10, 12, or 18 hours, 1, 2, 3, 4, 5, or 6 days, or 1, 2, 3, or 4 weeks prior to the administration of the therapeutic agent. In some embodiments, the IL-11 analog or a fragment or derivative thereof is administered after the therapeutic agent, e.g., 0.5, 1, 2, 3, 4, 5, 10, 12, or 18 hours, 1, 2, 3, 4, 5, or 6 days, or 1, 2, 3, or 4 weeks after the administration of the therapeutic agent. In some embodiments, the IL-11 analog or a fragment or derivative thereof and the therapeutic agent are administered concurrently but on different schedules, e.g., the IL-11 analog or a fragment or derivative thereof is administered daily while the therapeutic or anticancer agent is administered once a week, once every two weeks, once every three weeks, or once every four weeks.

Compositions within the scope of this invention include all compositions wherein the IL-11 analog or a fragment or derivative thereof of the present invention is contained in an amount which is effective to achieve its intended purpose. While individual needs vary, determination of optimal ranges of effective amounts of each component is within the skill of the art. Typically, the IL-11 analog or a fragment or derivative thereof may be administered to animals, e.g. humans, at a dose of about 3 to about 20 μg/kg body weight/day. In other embodiments, the dose is about 5 to about 20 μg/kg/day, about 5 to about 15 μg/kg/day, about 5 to about 10 μg/kg/day, or about 5 to about 7 μg/kg/day.

In some embodiments, the composition comprising the IL-11 analog or a fragment or derivative thereof is in unit dosage form, e.g., a single-use container, pill, capsule, or topical composition. In one embodiment, the unit dosage form comprises less than about 4.5 mg of the IL-11 analog or a fragment or derivative thereof, e.g., from about 0.01 mg to about 4 mg, from about 0.05 mg to about 2 mg, or from about 0.1 mg to about 1 mg.

In addition to administering the IL-11 analog or a fragment or derivative thereof as an isolated polypeptide, the polypeptides of the invention may be administered as part of a pharmaceutical preparation containing suitable pharmaceutically acceptable carriers comprising excipients and auxiliaries which facilitate processing of the polypeptides into preparations which can be used pharmaceutically. Preferably, the preparations contain from about 0.01 to 99 percent, preferably from about 0.25 to 75 percent of active polypeptide, together with the excipient.

In one embodiment, pharmaceutical compositions comprise excipients that stabilize the IL-11 analog polypeptide or a fragment or derivative thereof, thereby preventing degradation upon storage. In one embodiment, the pharmaceutical composition is in a dry form, e.g., lyophilized, in order to preserve the stability of the polypeptide. The dry composition is dissolved in a suitable liquid, e.g., water or saline, immediately prior to administration to an animal. In another embodiment, the pharmaceutical compositions are in liquid form. Examples of suitable pharmaceutical compositions for IL-11 include compositions comprising IL-11, glycine, and a cryoprotectant, and optionally a polysorbate, methionine, and a buffering agent (see U.S. Pat. Nos. 6,270,757; 7,033,992).

The pharmaceutical compositions of the invention may be administered to any animal which may experience the beneficial effects of the compounds of the invention. Foremost among such animals are mammals, e.g., humans, although the invention is not intended to be so limited. Other animals include veterinary animals (cows, sheep, pigs, horses, dogs, cats and the like). In one embodiment, the animal is a human or a monkey.

The pharmaceutical compositions may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The preferred route of administration is dependent on the disease or disorder to be treated, ameliorated, or prevented. For example, for stimulation of thrombopoiesis the preferred route of administration is subcutaneous. For treatment, amelioration or prevention of gastrointestinal inflammatory disorders, the preferred route of administration is topical. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

The present invention is also directed to kits, including pharmaceutical kits. The kits can comprise the IL-11 analog or a fragment or derivative thereof, as well appropriate controls, such as positive and/or negative controls. In some embodiments, the kits can comprise a pharmaceutical composition comprising the IL-11 analog. The kit preferably comprises additional components, such as, for example, instructions, solid support, reagents helpful for quantification, and the like. The compound or agent can be packaged in a suitable container.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Thrombopoietic Activity of IL-11 Analog in Nonhumans Primates

1. Study Design

Recombinant human IL-11 protein was administered as a daily subcutaneous injection for 7 days followed by 23-day washout period during each experimental cycle. Doses of 5 μg/kg/day was given in the first experimental cycle. Dose escalation was allowed if no adverse event was found. Doses of 15 and 50 μg/kg/day were administered in the second and third experimental cycles, respectively.

2. Materials & Methods

1) Administration of Recombinant Human IL-11 Protein

Three female cynomolgus monkeys (Macaca fascicularis) were administered saline, NEUMEGA®, or IL-11 analog, respectively. The recombinant human IL-11 proteins, NEUMEGA® and IL-11 analog, were diluted with saline to appropriate concentrations and subcutaneously injected for 7 days with the indicated doses.

2) Determination of Platelet Count

To determine the basal level of platelet count, blood samples were obtained from each animal four days before the start of treatment. Additional blood samples were taken three times a week throughout the study period. The number of platelets in blood was measured using complete blood counts.

3. Results and Discussion

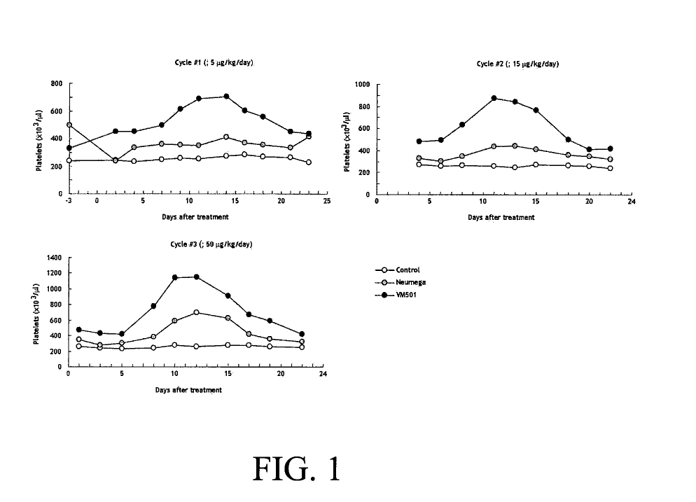

To determine the effects of the recombinant human IL-11 proteins, NEUMEGA® and IL-11 analog, on the level of platelets, blood samples were taken from each animal and analyzed by complete blood counts. The monkey treated with NEUMEGA® showed an increase of platelet counts over the basal level only at the 50 μg/kg/day dose (FIG. 1). The level of platelets peaked on day 12 and declined to the basal value on day 17 after treatment. The platelet counts on the peak showed an increase of 2.0×10⁵ cells/μl compared with the basal level. Administration of IL-11 analog provoked the increase of platelet counts over basal level at all doses (FIG. 1). The concentration of platelets reached the peak on day 11˜14 and gradually decreased. The net increases of 3.7, 5.4, 8.2×10⁵ cells/μl on the peak were observed at the doses 5, 15, and 50 μg/kg/day, respectively. There was no adverse event during the study period.

This study showed that IL-11 analog can exert more influence on the production of platelets at low dose compared with NEUMEGA®.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

1-24. (canceled)

25. A method of increasing the platelet count in an animal in need thereof, comprising administering to said animal an IL-11 analog comprising the sequence of SEQ ID NO:1 or a fragment or derivative thereof at a dose of about 3 to about 20 μg/kg/day.

26-27. (canceled)

28. The method of claim 25, wherein said IL-11 analog or a fragment or derivative thereof is administered at a dose of about 5 to about 10 μg/kg/day.

29. (canceled)

30. The method of claim 25, wherein said derivative comprises an amino acid sequence that is at least 70% identical to the amino acid sequence of SEQ ID NO:1.

31. The method of claim 25, wherein said derivative or fragment retains one or both of Ala10 and Asn134 of said IL-11 analog (corresponding to positions 1 and 125 of SEQ ID NO:1).

32. The method of claim 25, wherein said animal is a human or a monkey.

33. A pharmaceutical composition comprising an IL-11 analog comprising the sequence of SEQ ID NO: 1 or a fragment or derivative thereof and a pharmaceutically acceptable carrier.

34. The pharmaceutical composition of claim 33, in unit dosage form.

35. The pharmaceutical composition of claim 34, wherein said unit dosage form comprises less than about 4.5 mg of said IL-11 analog or a fragment or derivative thereof.

36-40. (canceled)

41. The pharmaceutical composition of claim 33, wherein said derivative comprises an amino acid sequence that is at least 70% identical to the amino acid sequence of SEQ ID NO:1.

42. The pharmaceutical composition of claim 33, wherein said derivative or fragment retains one or both of Ala10 and Asn134 of said IL-11 analog (corresponding to positions 1 and 125 of SEQ ID NO:1).

43. A method of preparing a pharmaceutical composition comprising an IL-11 analog comprising the sequence of SEQ ID NO:1 or a fragment or derivative thereof, the method comprising admixing said IL-11 analog or a derivative thereof and a pharmaceutically acceptable carrier.

44. The method of claim 43, wherein prior to admixing, said IL-11 analog or a fragment or derivative thereof is in a unit dosage form comprising less than about 4.5 mg of said IL-11 analog or a fragment or derivative thereof.

45-49. (canceled)

50. The method of claim 43, wherein said derivative comprises an amino acid sequence that is at least 90% identical to the amino acid sequence of SEQ ID NO 1.

51. The method of claim 43, wherein said derivative or fragment retains one or both of Ala10 and Asn134 of the IL-11 analog (corresponding to positions 1 and 125 of SEQ ID NO:1).

52. The method of claim 25 wherein said derivative or fragment comprises a polypeptide comprising amino acids x to 169 of SEQ ID NO:1, wherein x is any integer from 1 to 40, and wherein said analog retains substantially all of the biological activity of the full-length IL-11 analog.

53. The method of claim 25 wherein said derivative or fragment comprises a polypeptide comprising amino acids 1 to y of SEQ ID NO:1, wherein in y is any integer from 125 to 169, and wherein said analog retains substantially all of the biological activity of the full-length IL-11 analog.

54. The method of claim 25 wherein said derivative or fragment comprises a polypeptide comprising amino acids x to y of SEQ ID NO:1, wherein x is any integer from 1 to 40 and y is any integer from 125 to 169, and wherein said analog retains substantially all of the biological activity of the full-length IL-11 analog.

55. The method or pharmaceutical composition of claim 54, wherein x is an integer from 1 to 25.

56. (canceled)

57. The method or pharmaceutical composition of claim 54, wherein x is an integer from 1 to 5.

58. The method or pharmaceutical composition of claim 54, wherein y is an integer from 140 to 169.

59. (canceled)

60. The method or pharmaceutical composition of claim 54, wherein y is an integer from 165 to 169.
